SEQUENCE LISTING (39 sequences)

SEQ ID NO: 1 - protein, 20 aa, Homo Sapiens
APDTRPAPGSTAPPAHGVTS

SEQ ID NO: 2 - protein, 20 aa, Homo Sapiens
APATEPASGSAATWGQDVTS

SEQ ID NO: 3 - protein, 20 aa, Homo Sapiens
VPVTRPALGSTTPPAHDVTS

SEQ ID NO: 4 - protein, 20 aa, Homo Sapiens
APDNKPAPGSTAPPAHGVTS

SEQ ID NO: 5 - protein, 20 aa, Homo Sapiens
APDNRPALGSTAPPVHNVTS

SEQ ID NO: 6 - protein, 9 aa, Homo Sapiens
FLSFHISNL

SEQ ID NO: 7 - protein, 21 aa, Homo Sapiens
NSSLEDPSTDYYQELQRDISE

SEQ ID NO: 8 - protein, 9 aa, Homo Sapiens
NLTISDVSV

SEQ ID NO: 9 - DNA, 1927 bp, Artificial Sequence
  Sequence of M. tuberculosis HSP70 from the expression vector JNW266
cggaccggccaccatggctagcgaattcggcgcgccagctagcatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcggtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggcggcgagccgggcggtgcccaccccggctcggctgatgacgttgtggacgcggaggtggtcgacgacggccgggaggccaagtgactcgag

SEQ ID NO: 10 - DNA, 3694 bp, Artificial Sequence
  DNA sequences of the MUC1-HSP70 fusions encoded by the plasmids JNW661 and JNW663
cggaccggccaccatggctagaacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggcccgccccgggctccaccgcccccccagcccacggtgtcacctcggccccggacaccaggcccgccccgggctccaccgcgcccgcagcccacggtgtcacctcggccccggacaccaggccggccccgggctccaccgccccccaagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtctagcatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcggtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcytcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggcggcgagccgggcggtgcccaccccggctcggctgatgacgttgtggacgcggaggtggtcgacgacggccgggaggccaagtgactcgag

SEQ ID NO: 11 - DNA, 1939 bp, Artificial Sequence
  DNA sequences of the 3′ modified HSP70 expression constructs JNW716 and JNW719.
gctagacggaccggccaccatggctagcgaattcggcgcgccagctagcatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcggtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggcggcgagccgggcggtgcccaccccggctcggctgatgacgttgtggacgcggaggtggtcgacgacggccgggaggccaagtctagatgactcgag

SEQ ID NO: 12 - DNA, 1981 bp, Artificial Sequence
  DNA sequences of the 3′ modified HSP70 expression constructs JNW716 and JNW719.
gctagacggaccggccaccatggctagcacaccgggcacccagtctcctttcttcctgctgctgctcctcacagtgcttacagttgctagcatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcggtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggcggcgagccgggcggtgcccaccccggctcggctgatgacgttgtggacgcggaggtggtcgacgacggccgggaggccaagtctagatgactcgag

SEQ ID NO: 13 - DNA, 3727 bp, Artificial Sequence
  DNA sequences of the HSP70-MUC1 expression constructs JNW722, JNW723, JNW725 and JNW727
gctagacggaccggccaccatggctagcgaattcggcgcgccagctagcatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcggtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggcggcgagccgggcggtgcccaccccggctcggctgatgacgttgtggacgcggaggtggtcgacgacggccgggaggccaagtctagaacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggcccgccccgggctccaccgcccccccagcccacggtgtcacctcggccccggacaccaggcccgccccgggctccaccgcgcccgcagcccacggtgtcacctcggccccggacaccaggccggccccgggctccaccgccccccaagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtctagatgactcgag

SEQ ID NO: 14 - DNA, 3670 bp, Artificial Sequence
  DNA sequences of the HSP70-MUC1 expression constructs JNW722, JNW723, JNW725 and JNW727
gctagacggaccggccaccatggctagcgaattcggcgcgccagctagcatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcggtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggcggcgagccgggcggtgcccaccccggctcggctgatgacgttgtggacgcggaggtggtcgacgacggccgggaggccaagtctagagttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggcccgccccgggctccaccgcccccccagcccacggtgtcacctcggccccggacaccaggcccgccccgggctccaccgcgcccgcagcccacggtgtcacctcggccccggacaccaggccggccccgggctccaccgccccccaagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtctagatgactcgag

SEQ ID NO: 15 - DNA, 3769 bp, Artificial Sequence
  DNA sequences of the HSP70-MUC1 expression constructs JNW722, JNW723, JNW725 and JNW727
gctagacggaccggccaccatggctagcacaccgggcacccagtctcctttcttcctgctgctgctcctcacagtgcttacagttgctagcatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcggtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggcggcgagccgggcggtgcccaccccggctcggctgatgacgttgtggacgcggaggtggtcgacgacggccgggaggccaagtctagaacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggcccgccccgggctccaccgcccccccagcccacggtgtcacctcggccccggacaccaggcccgccccgggctccaccgcgcccgcagcccacggtgtcacctcggccccggacaccaggccggccccgggctccaccgccccccaagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtctagatgactcgag

SEQ ID NO: 16 - DNA, 3712 bp, Artificial Sequence
  DNA sequences of the HSP70-MUC1 expression constructs JNW722, JNW723, JNW725 and JNW727
gctagacggaccggccaccatggctagcacaccgggcacccagtctcctttcttcctgctgctgctcctcacagtgcttacagttgctagcatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcggtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggcggcgagccgggcggtgcccaccccggctcggctgatgacgttgtggacgcggaggtggtcgacgacggccgggaggccaagtctagagttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggcccgccccgggctccaccgcccccccagcccacggtgtcacctcggccccggacaccaggcccgccccgggctccaccgcgcccgcagcccacggtgtcacctcggccccggacaccaggccggccccgggctccaccgccccccaagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtctagatgactcgag

SEQ ID NO: 17 - DNA, 18 bp, Artificial Sequence
  Oligonucleotide primers
gtgtcgcttgaccgagcg

SEQ ID NO: 18 - DNA, 18 bp, Artificial Sequence
  Oligonucleotide primers
ggcgtccttggtggcctg

SEQ ID NO: 19 - DNA, 18 bp, Artificial Sequence
  Oligonucleotide primers
ctggtcccagtcgtcgcc

SEQ ID NO: 20 - DNA, 18 bp, Artificial Sequence
  Oligonucleotide primers
cgggttcttgtcggcgtc

SEQ ID NO: 21 - DNA, 19 bp, Artificial Sequence
  Oligonucleotide primers
gggttccttgccgccggtg

SEQ ID NO: 22 - DNA, 18 bp, Artificial Sequence
  Oligonucleotide primers
ggttgtcgtcggcggtgg

SEQ ID NO: 23 - DNA, 19 bp, Artificial Sequence
  Oligonucleotide primers
cttgccggtgcccttgtcc

SEQ ID NO: 24 - DNA, 19 bp, Artificial Sequence
  Oligonucleotide primers
accaccctcggcctcacgc

SEQ ID NO: 25 - DNA, 19 bp, Artificial Sequence
  Oligonucleotide primers
ttgccccagagcctgcgac

SEQ ID NO: 26 - DNA, 18 bp, Artificial Sequence
  Oligonucleotide primers
agtcgcaggctctggggc

SEQ ID NO: 27 - DNA, 18 bp, Artificial Sequence
  Oligonucleotide primers
gcgtgaggccgagggtgg

SEQ ID NO: 28 - DNA, 19 bp, Artificial Sequence
  Oligonucleotide primers
gacaagggcaccggcaagg

SEQ ID NO: 29 - DNA, 18 bp, Artificial Sequence
  Oligonucleotide primers
ccaccgccgacgacaacc

SEQ ID NO: 30 - DNA, 18 bp, Artificial Sequence
  Oligonucleotide primers
accggcggcaaggaaccc

SEQ ID NO: 31 - DNA, 18 bp, Artificial Sequence
  Oligonucleotide primers
gacgccgacaagaacccg

SEQ ID NO: 32 - DNA, 19 bp, Artificial Sequence
  Oligonucleotide primers
ggcgacgactgggaccagc

SEQ ID NO: 33 - DNA, 18 bp, Artificial Sequence
  Oligonucleotide primers
aggccaccaaggacgccg

SEQ ID NO: 34 - DNA, 18 bp, Artificial Sequence
  Oligonucleotide primers
cgctcggtcaagcgacac

SEQ ID NO: 35 - DNA, 38 bp, Artificial Sequence
  Oligonucleotide primers
gcgaacgcgtctcgagtcacttggcctcccggccgtcg

SEQ ID NO: 36 - DNA, 76 bp, Artificial Sequence
  Oligonucleotide primers
gcgcatctagacggaccggccaccatggctagcgaattcggcgcgccagctagcatggctcgtgcggtcgggatcg

SEQ ID NO: 37 - DNA, 22 bp, Artificial Sequence
  Oligonucleotide primers
gcttgatgttaccccgctgagc

SEQ ID NO: 38 - DNA, 41 bp, Artificial Sequence
  Oligonucleotide primers
gacgctcgagtcatctagacttggcctcccggccgtcgtcg

SEQ ID NO: 39 - DNA, 126 bp, Artificial Sequence
  Oligonucleotide primers
ctagcacaccgggcacccagtctcctttcttcctgctgctgctcctcacagtgcttacagttgctagcaactgtaagcactgtgaggagcagcagcaggaagaaaggagactgggtgcccggtgtg

(19) United States
(12) Patent Application Publication
(10) Pub. No.: US 2007/0042047 A1
(43) Pub. Date: Feb. 22, 2007

(54) Vaccines

(75) Inventors: Paul Andrew Hamblin, Hertfordshire (GB); Maria de los Angeles Rocha Del Cura, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 10/571,972

(22) PCT Filed: Sep. 13, 2004
(86) PCT No.: PCT/EP04/10323

§ 371 Date: Mar. 15, 2006

(30) Foreign Application Priority Data
Sep. 15, 2003 (GB) 0321614.0

(51) Int. Cl. A61K 48/00; C07K 14/195; C07H 21/04; C12P 21/06

(52) U.S. Cl. 424/489; 514/44; 435/69.1; 435/320.1; 435/325; 530/350; 536/23.5; 977/906

(57) ABSTRACT

The present invention relates to the novel nucleic acid constructs, useful in nucleic acid vaccination protocols for the treatment and prophylaxis of MUC-1 expressing tumours. In particular, the construct comprises a fusion between a heat shock protein gene HSP70, typically from Mycobacterium tuberculosis and MUCl-1 or derivative thereof. The invention further provides pharmaceutical compositions comprising said constructs and proteins, particularly pharmaceutical compositions adapted for particle mediated delivery, methods for producing them, and their use in medicine, particularly in the treatment of MUCl-1 expressing tumours.

The present invention relates to the novel nucleic acid constructs, useful in nucleic acid vaccination protocols for the treatment and prophylaxis of MUC-1 expressing tumours. In particular, the invention further pertains to novel proteins encoded by such constructs. In particular the construct comprises a fusion between a heat shock protein gene HSP70, typically from Mycobacterium tuberculosis and MUC-1 or derivative thereof. The invention further provides pharmaceutical compositions comprising said constructs and proteins, particularly pharmaceutical compositions adapted for particle mediated delivery, methods for producing them, and their use in medicine, particularly in the treatment of MUC-1 expressing tumours.

### BACKGROUND TO THE INVENTION

The epithelial cell mucin MUC-1 (also known as episialin or polymorphic epithelial mucin, PEM) is a large molecular-weight glycoprotein expressed on many epithelial cells. The protein consists of a cytoplasmic tail, a transmembrane domain and a variable number of tandem repeats of a 20 amino acid motif (herein termed the VNTR monomer, it may also be known as the VNTR epitope, or the VNTR repeat) containing a high proportion of proline, serine and threonine residues. The number of repeats is variable due to genetic polymorphism at the MUC-1 locus, and most frequently lies within the range 30-100 (Swallow et al, 1987, Nature 328:82-84). In normal ductal epithelia, the MUC-1 protein is found only on the apical surface of the cell, exposed to the duct lumen (Graham et al, 1996, Cancer Immunol Immunother 42:71-80; Barratt-Boyes et al, 1996, Cancer Immunol Immunother 43:142-151). One of the most striking features of the MUC-1 molecule is its extensive O-linked glycosylation. There are five O-linked glycosylation sites available within each MUC-1 VNTR monomer.

The VNTR can be characterised as typical or perfect repeats and imperfect (atypical) repeats which has minor variation for the perfect repeat comprising two to three differences over the 20 amino acids. The following is the sequence of the perfect repeat.

```
1 2 3 4 5 6 7 8 9 10 11 12 13 14 15 16 17 18 19 20
A P D T R P A P G S T A P P A H G V T S
                E S T
                                                            A
                                                            Q
```

Amino acids that are underlined may be substituted for the amino acid residues shown.

Imperfect repeats have different amino acid substitutions to the consensus sequence above with 55-90% identity at the amino acid level. The four imperfect repeats are shown below, with the substitutions underlined:

APDTRPAPGSTAPPAHGVTS - perfect repeat
APATEPASGSAATWGQDVTS - imperfect repeat 1
VPVTRPALGSTTPPAHDVTS - imperfect repeat 2
APDNKPAPGSTAPPAHGVTS - imperfect repeat 3
APDNRPALGSTAPPVHNVTS - imperfect repeat 4

The imperfect repeat in wild type—Muc-1 flank the perfect repeat region. In malignant carcinomas arising by neoplastic transformation of these epithelial cells, several changes affect the expression of MUC-1. The polarised expression of the protein is lost, and it is found spread over the whole surface of the transformed cell. The total amount of MUC-1 is also increased, often by 10-fold or more (Strous & Dekker, 1992, Crit Rev Biochem Mol Biol 27:57-92). Most significantly, the quantity and quality of the O-linked carbohydrate chains changes markedly. Fewer serine and threonine residues are glycosylated. Those carbohydrate chains that are found are abnormally shortened, creating the tumour-associated carbohydrate antigen STn (Lloyd et al, 1996, J Biol Chem, 271:33325-33334). As a result of these glycosylation changes, various epitopes on the peptide chain of MUC-1 which were previously screened by the carbohydrate chains become accessible. One epitope which becomes accessible in this way is formed by the sequence APDTR (Ala 8-Arg 12) present in each 20 amino acid VNTR perfect monomer (Burchell et al, 1989, Int J Cancer 44:691-696).

It is apparent that these changes in MUC-1 mean that a vaccine that can activate the immune system against the form of MUC-1 expressed on tumours may be effective against epithelial cell tumours, and indeed other cell types where MUC-1 is found, such as T cell lymphocytes. One of the main effector mechanisms used by the immune system to kill cells expressing abnormal proteins is a cytotoxic T lymphocyte immune response (CTL's) and this response is desirable in a vaccine to treat tumours, as well as an antibody response. A good vaccine will activate all arms of the immune response. However, current carbohydrate and peptide vaccines such as Theratope or BLP25 (Biomira Inc, Edmonton, Canada) preferentially activate one arm of the immune response—a humoral and cellular response respectively, and better vaccine designs are desirable to generate a more balanced response.

Nucleic acid vaccines provide a number of advantages over conventional protein vaccination, in that they are easy to produce in large quantity. Even at small doses they have been reported to induce strong immune responses, and can induce a cytotoxic T lymphocyte immune response as well as an antibody response.

Heat shock proteins (HSPs) are a member of a group of proteins more generally known as stress proteins and have many functions essential for cellular survival. They participate in both innate and adaptive immune responses through their ability to interact with a wide range of proteins and peptides. HSPs are widely conserved and present in diverse organisms, such as the protozoan Plasmodium falciparum, bacteria such as E. coli, Mycobacteria and in higher organisms. In bacteria, the major stress proteins are HSP60 and HSP70 and accumulate at very high levels (upto 25%) in stressed cells, whilst in normal settings will account for less than 5% of cell protein. HSPs can be grouped into one of 10 families, with each family consisting of 1-5 closely related members (see Srivastava, Nature Reviews Immunology (2002) 2:185-194 for an extensive review). Some of the main families of HSPs include the HSP60 group (HSP60, HSP65, GROEL), the HSP70 group (DNAK/HSP70, HSP72/73/110, GRP78/170), the HSP90 group (gp96, HSP86, HTPG, HSC84) and the small HSPs group (HSP10/16/20/25/26/27, GROES, alpha-crystallin).

U.S. Pat. No. 6,335, 183 discloses methods of modulating an individuals immune response by the use if bacterial stress proteins. Fusion compositions comprising such stress proteins and HIV gag are mentioned.

### SUMMARY OF THE INVENTION

The present invention provides a nucleic acid molecule encoding a MUC-1 protein or derivative which is capable of raising an immune response in vivo, said immune response being capable of recognising a MUC-1 expressing tumour, wherein the molecule additionally encodes a heat shock protein (HSP) or fragment thereof capable of modifying the immune response to the MUC-1 component. It is preferred that the fragment contain domain II from the ATPase domain of the HSP.

In one embodiment, the nucleic acid encodes for a MUC-1 derivative as described above devoid of any repeat (both perfect and imperfect) units.

In an alternative embodiment, the nucleic acid sequence is devoid of only the perfect repeats. In yet a further embodiment, the nucleic acid construct contains between 1 and 15 perfect repeats, preferably 7 perfect repeats.

In an embodiment of the invention, the MUC-1 derivative maybe codon modified from wild type MUC-1. In particular, the non-perfect repeat region in a more preferred embodiment has a RSCU (Relative synomous Codon Usage or Codin Index Cl) of at least 0.65 and less than 80% identity to the non-perfect repeat region.

Such constructs are capable of raising both a cellular and also an antibody response that recognise MUC-1 expressing tumour cells. Fusion to HSP improves the kinetics and functionality of the immune response to MUC-1.

The constructs can also contain altered repeat (VNTR units) such as reduced glycosylation mutants. Foreign T-cell epitopes that may be incorporated include T-helper epitopes such as derived from bacterial proteins and toxins and from viral sources, eg. T-Helper epitopes from Diphtheria or Tetanus, eg P2 and P30 or epitopes from Hep B case antigen. These maybe incorporated within or at either end of the MUC-1 constructs of the invention.

The heat shock protein is typically a bacterial, typically an E. coli or Mycobacterium protein, preferably HSP70 more preferably HSP70 from Mycobacterium Tuberculosis. Members of the HSP70 group include DNAK.HSP70, HSP72/73/110, GRP78/170. Other HSP proteins contemplated for use in the present invention include those from the HSP60 group (HSP60, HSP65 GROEL), the HSP90 group and the small HSPs group.

In further aspect of the invention the nucleic acid sequence is a DNA sequence in the form of a plasmid. Preferably the plasmid is super-coiled.

Proteins encoded by the nucleotide molecules of the invention are novel and form an aspect of the invention.

In a further aspect of the invention there is provided a pharmaceutical composition comprising a nucleic acid sequence or protein as herein described and a pharmaceutical acceptable excipient, diluent or carrier.

Preferably for nucleic acid administration the carrier is a gold bead and the pharmaceutical composition is amenable to delivery by particle mediated drug delivery.

In yet a further embodiment, the invention provides the pharmaceutical composition and nucleic acid constructs for use in medicine. In particular, there is provided a nucleic acid construct of the invention, in the manufacture of a medicament for use in the treatment or prophylaxis of MUC-1 expressing tumours.

The invention further provides for methods of treating a patient suffering from or susceptible to a MUC-1 expressing tumour, particularly carcinoma of the breast, lung, (particularly non-small cell lung carcinoma), prostate, gastric and other GI (gastrointestinal) carcinomas by the administration of a safe and effective amount of a composition nucleic acid or protein as herein described.

In yet a further embodiment the invention provides a method of producing a pharmaceutical composition as herein described by admixing a nucleic acid construct, plasmid or protein of the invention with a pharmaceutically acceptable excipient, diluent or carrier.

### DETAILED DESCRIPTION OF THE INVENTION

The present invention provides a nucleic acid molecule encoding a MUC-1 protein or derivative thereof which is capable of recognising a MUC-1 expressing tumour wherein the sequence additionally encodes a heat shock protein or fragment thereof capable of modifying the immune response to the MUC-1 component.

Preferably, the heat shock protein is HSP from a Mycobacterium typically Mycobacterium tuberculosis, more typically Mycobacterium tuberculosis HSP70.

The HSP70 maybe fused to either end of MUC-1 molecule, but it is preferred that the MUC-1 component be at the C terminus as such proteins are more stable. If the construct includes the MUC-1 signal sequence, this may be placed at the N terminus of the HSP.

The MUC-1 component may include the full length wild type gene, but it is preferable to use a shorter derivative with less than 15 VNTR units.

The wild type MUC-1 molecule contains a signal sequence, a leader sequence, imperfect or atypical VNTR, the perfect VNTR region, a further atypical VNTR, a non-VNTR extracellular domain a transmembrane domain and a cytoplasmic domain.

The non-VNTR extracellular domain is approximately 80 amino acids, 5′ of VNTR and 190-200 amino acids 3′ VNTR. All constructs of the invention comprise at least one epitope from this region. An epitope is typically formed from at least seven amino acid sequence. Accordingly the constructs of the present invention include at least one epitope from the non VNTR extra-cellular domain. Preferably substantially all or more preferably all of the non-VNTR domain is included. It is particularly preferred that construct contains the epitope comprised by the sequence FLSFHISNL; NSSLEDPSTDYYQELQRDISE, or NLTISDVSV. More preferred is that two, preferable all three, epitope sequences are incorporated in the construct.

In a preferred embodiment the constructs comprise an N-terminal leader sequence. The signal sequence, transmembrane domain and cytoplasmic domain are individually all optionally present or deleted. When present it is preferred that all these regions are modified.

Preferred constructs according to the invention are:
1) HSP70-MUC-1 (ie Full MUC-1 with no perfect repeats)
2) HSP70-MUC-1 Δss (As I, but also devoid of signal sequence)
3) HSP70-MUC-1 ΔTM ΔCYT (As 1, but devoid of Transmembrane and cytoplasmic domains)
4) HSP70-MUC-1 Δss ΔTM ΔCYT (As 3, but also devoid of signal sequence)

Also preferred are equivalent constructs of 1 to 4 above, but devoid of imperfect MUC-1 repeat units. Such constructs are referred to as HSP-gutted-MUC-1.

In an embodiment one or more of the imperfect VNTR units is mutated to reduce the potential for glycosylation, by altering a glycosylation site. The mutation is preferably a replacement, but can be an insertion or a deletion. Typically at least one threonine or seriene is substituted with valine, Isoleucine, alanine, asparagine, phenylalanine or tryptophan. It is thus preferred that at least one, preferably 2 or 3 or more are substituted with an amino acid as noted above.

Other preferred constructs are the equivalent to the above, but comprising from 1-15, preferably 2-8, most preferably 7 VNTR (perfect) repeat units.

In a further embodiment, the gutted MUC-1 nucleic acid is provided with a restriction site at the junction of the leader sequence and the extracellular domain. Typically this restriction site is a Nhe1 site. This can be utilised as a cloning site to insert sequences encoding for other peptides including, for example glycosylation mutants (ie. VNTR regions mutated to remove O-glycosylation sites), or heterologous sequences that encode T-Helper epitopes such as P2 or P30 from Tetanus toxin, or wild type VNTR units.

The DNA code has 4 letters (A, T, C and G) and uses these to spell three letter “codons” which represent the amino acids the proteins encodes in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons.

Where more than one codon is available to code for a given amino acid, it has been observed that the codon usage patterns of organisms are highly non-random. Different species show a different bias in their codon selection and, furthermore, utilisation of codons may be markedly different in a single species between genes which are expressed at high and low levels. This bias is different in viruses, plants, bacteria and mammalian cells, and some species show a stronger bias away from a random codon selection than others. For example, humans and other mammals are less strongly biased than certain bacteria or viruses. For these reasons, there is a significant probability that a mammalian gene expressed in E. coli or a viral gene expressed in mammalian cells will have an inappropriate distribution of codons for efficient expression. It is believed that the presence in a heterologous DNA sequence of clusters of codons which are rarely observed in the host in which expression is to occur, is predictive of low heterologous expression levels in that host.

In consequence, codons preferred by a particular prokaryotic (for example E. coli or yeast) or eucaryotic host can be modified so as to encode the same protein, but to differ from a wild type sequence. The process of codon modification may include any sequence, generated either manually or by computer software, where some or all of the codons of the native sequence are modified. Several method have been published (Nakamura et.al., Nucleic Acids Research 1996, 24:214-215; WO98/34640). One preferred method according to this invention is Syngene method, a modification of Calcgene method (R. S. Hale and G Thompson (Protein Expression and Purification Vol.12 pp.185-188 (1998)). This process of codon modification may have some or all of the following benefits: 1) to improve expression of the gene product by replacing rare or infrequently used codons with more frequently used codons, 2) to remove or include restriction enzyme sites to facilitate downstream cloning and 3) to reduce the potential for homologous recombination between the insert sequence in the DNA vector and genomic sequences and 4) to improve the immune response in humans. The sequences of the present invention advantageously have reduced recombination potential, but express to at least the same level as the wild type sequences. Due to the nature of the algorithms used by the SynGene programme to generate a codon modified sequence, it is possible to generate an extremely large number of different codon modified sequences which will perform a similar function. In brief, the codons are assigned using a statistical method to give synthetic gene having a codon frequency closer to that found naturally in highly expressed human genes such as β-Actin.

In an embodiment of the invention the polynucleotides of the present invention, the codon usage pattern is altered from that typical of MUC-1 to more closely represent the codon bias of the target highly expressed human gene. The “codon usage coefficient” is a measure of how closely the codon pattern of a given polynucleotide sequence resembles that of a target species. Codon frequencies can be derived from literature sources for the highly expressed genes of many species (see e.g. Nakamura et.al. Nucleic Acids Research 1996, 24:214-215). The codon frequencies for each of the 61 codons (expressed as the number of occurrences occurrence per 1000 codons of the selected class of genes) are normalised for each of the twenty natural amino acids, so that the value for the most frequently used codon for each amino acid is set to 1 and the frequencies for the less common codons are scaled to lie between zero and 1. Thus each of the 61 codons is assigned a value of 1 or lower for the highly expressed genes of the target species. In order to calculate a codon usage coefficient for a specific polynucleotide, relative to the highly expressed genes of that species, the scaled value for each codon of the specific polynucleotide are noted and the geometric mean of all these values is taken (by dividing the sum of the natural logs of these values by the total number of codons and take the anti-log). The coefficient will have a value between zero and 1 and the higher the coefficient the more codons in the polynucleotide are frequently used codons. If a polynucleotide sequence has a codon usage coefficient of 1, all of the codons are “most frequent” codons for highly expressed genes of the target species.

According to the present invention, the codon usage pattern of the polynucleotide will preferably exclude codons representing <10% of the codons used for a particular amino acid. A relative synonymous codon usage (RSCU) value is the observed number of codons divided by the number expected if all codons for that amino acid were used equally frequently. A polynucleotide of the present invention will preferably exclude codons with an RSCU value of less than 0.2 in highly expressed genes of the target organism. A polynucleotide of the present invention will generally have a codon usage coefficient for highly expressed human genes of greater than 0.6, preferably greater than 0.65, most preferably greater than 0.7. Codon usage tables for human can also be found in Genbank.

In comparison, a highly expressed beta actin gene has a RSCU of 0.747.

The codon usage table for a homo sapiens is set out below:

Codon usage for human (highly expressed)
genes 1/24/91 (human_high.cod)

| | | | | |
|---|---|---|---|---|
| AmAcid | Codon | Number | /1000 | Fraction |

| | | | | |
|---|---|---|---|---|
| Gly | GGG | 905.00 | 18.76 | 0.24 |
| Gly | GGA | 525.00 | 10.88 | 0.14 |
| Gly | GGT | 441.00 | 9.14 | 0.12 |
| Gly | GGC | 1867.00 | 38.70 | 0.50 |
| Glu | GAG | 2420.00 | 50.16 | 0.75 |
| Glu | GAA | 792.00 | 16.42 | 0.25 |
| Asp | GAT | 592.00 | 12.27 | 0.25 |
| Asp | GAC | 1821.00 | 37.75 | 0.75 |
| Val | GTG | 1866.00 | 38.68 | 0.64 |
| Val | GTA | 134.00 | 2.78 | 0.05 |
| Val | GTT | 198.00 | 4.10 | 0.07 |
| Val | GTC | 728.00 | 15.09 | 0.25 |
| Ala | GCG | 652.00 | 13.51 | 0.17 |
| Ala | GCA | 488.00 | 10.12 | 0.13 |
| Ala | GCT | 654.00 | 13.56 | 0.17 |
| Ala | GCC | 2057.00 | 42.64 | 0.53 |
| Arg | AGG | 512.00 | 10.61 | 0.18 |
| Arg | AGA | 298.00 | 6.18 | 0.10 |
| Ser | AGT | 354.00 | 7.34 | 0.10 |
| Ser | AGC | 1171.00 | 24.27 | 0.34 |
| Lys | AAG | 2117.00 | 43.88 | 0.82 |
| Lys | AAA | 471.00 | 9.76 | 0.18 |
| Asn | AAT | 314.00 | 6.51 | 0.22 |
| Asn | AAC | 1120.00 | 23.22 | 0.78 |
| Met | ATG | 1077.00 | 22.32 | 1.00 |
| Ile | ATA | 88.00 | 1.82 | 0.05 |
| Ile | ATT | 315.00 | 6.53 | 0.18 |
| Ile | ATC | 1369.00 | 28.38 | 0.77 |
| Thr | ACG | 405.00 | 8.40 | 0.15 |
| Thr | ACA | 373.00 | 7.73 | 0.14 |
| Thr | ACT | 358.00 | 7.42 | 0.14 |
| Thr | ACC | 1502.00 | 31.13 | 0.57 |
| Trp | TGG | 652.00 | 13.51 | 1.00 |
| End | TGA | 109.00 | 2.26 | 0.55 |
| Cys | TGT | 325.00 | 6.74 | 0.32 |
| Cys | TGC | 706.00 | 14.63 | 0.68 |
| End | TAG | 42.00 | 0.87 | 0.21 |
| End | TAA | 46.00 | 0.95 | 0.23 |
| Tyr | TAT | 360.00 | 7.46 | 0.26 |
| Tyr | TAC | 1042.00 | 21.60 | 0.74 |
| Leu | TTG | 313.00 | 6.49 | 0.06 |
| Leu | TTA | 76.00 | 1.58 | 0.02 |
| Phe | TTT | 336.00 | 6.96 | 0.20 |
| Phe | TTC | 1377.00 | 28.54 | 0.80 |
| Ser | TCG | 325.00 | 6.74 | 0.09 |
| Ser | TCA | 165.00 | 3.42 | 0.05 |
| Ser | TCT | 450.00 | 9.33 | 0.13 |
| Ser | TCC | 958.00 | 19.86 | 0.28 |
| Arg | CGG | 611.00 | 12.67 | 0.21 |
| Arg | CGA | 183.00 | 3.79 | 0.06 |
| Arg | CGT | 210.00 | 4.35 | 0.07 |
| Arg | CGC | 1086.00 | 22.51 | 0.37 |
| Gln | CAG | 2020.00 | 41.87 | 0.88 |
| Gln | CAA | 283.00 | 5.87 | 0.12 |
| His | CAT | 234.00 | 4.85 | 0.21 |
| His | CAC | 870.00 | 18.03 | 0.79 |
| Leu | CTG | 2884.00 | 59.78 | 0.58 |
| Leu | CTA | 166.00 | 3.44 | 0.03 |
| Leu | CTT | 238.00 | 4.93 | 0.05 |
| Leu | CTC | 1276.00 | 26.45 | 0.26 |
| Pro | CCG | 482.00 | 9.99 | 0.17 |
| Pro | CCA | 456.00 | 9.45 | 0.16 |
| Pro | CCT | 568.00 | 11.77 | 0.19 |
| Pro | CCC | 1410.00 | 29.23 | 0.48 |

Accordingly in a preferred embodiment the polynucleotides of the invention are modified to more closely resemble the usage of a highly expressed human gene, such as β actin.

It is preferred that the non-VNTR units of the MUC-1 component are codon modified. The VNTR units when present may or may not be modified. The codon-modified sequence will preferably be less than 80% identical to the corresponding non-VNTR unit of Muc-1. The HSP component can, but need not be modified.

When comparing polynucleotide sequences, two sequences are said to be “identical” if the sequence of nucleotides in the two sequences is the same when aligned for maximum correspondence, as described below.

Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Thus in the present invention, the non-repeat region of the codon-modified and the non-repeat region of optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides of the invention. Software for performing BLAST analyses is publicly available through the National Center for Biotechnology Information.

According to a further aspect of the invention, an expression vector is provided which comprises and is capable of directing the expression of a polynucleotide sequence according to the invention. The vector may be suitable for driving expression of heterologous DNA in bacterial insect or mammalian cells, particularly human cells.

According to a further aspect of the invention, a host cell comprising a polynucleotide sequence according to the invention, or an expression vector according the invention is provided. The host cell may be bacterial, e.g. E. coli, mammalian, e.g. human, or may be an insect cell. Mammalian cells comprising a vector according to the present invention may be cultured cells transfected in vitro or may be transfected in vivo by administration of the vector to the mammal.

Proteins encoded by the nucleotide of the invention are also included as part of the present invention. The present invention further provides a pharmaceutical composition comprising a polynucleotide sequence according to the invention. Preferably the composition comprises a DNA vector. In preferred embodiments the composition comprises a plurality of particles, preferably gold particles, coated with DNA comprising a vector encoding a polynucleotide sequence of the invention which the sequence encodes a MUC-1 amino acid sequence as herein described. In alternative embodiments, the composition comprises a pharmaceutically acceptable excipient and a DNA vector according to the present invention.

Alternatively, a pharmaceutical composition comprising a protein of the invention and a pharmaceutically acceptable excipient. The composition may also include an adjuvant, or be administered either concomitantly with or sequentially with an adjuvant or immuno-stimulatory agent.

Thus it is an embodiment of the invention that the nucleotides, vectors or proteins of the invention be utilised with an adjuvant or immunostimulatory agent. In the case of nucleic acid administration it is preferred that the immunostimulatory agent is administered at the same time as the nucleic acid vector of the invention and in preferred embodiments are formulated together. Such immunostimulatory agents include, (but this list is by no means exhaustive and does not preclude other agents): synthetic imidazoquinolines such as imiquimod [S-26308, R-837], (Harrison, et al. ‘Reduction of recurrent HSV disease using imiquimod alone or combined with a glycoprotein vaccine’, Vaccine 19:1820-1826, (2001)); and resiquimod [S-28463, R-848] (Vasilakos, et al. ‘Adjuvant activites of immune response modifier R-848: Comparison with CpG ODN’, Cellular immunology 204: 64-74 (2000).), Schiff bases of carbonyls and amines that are constitutively expressed on antigen presenting cell and T-cell surfaces, such as tucaresol (Rhodes, J. et al. ‘Therapeutic potentiation of the immune system by costimulatory Schiff-base-forming drugs’, Nature 377: 71-75 (1995)), cytokine, chemokine and co-stimulatory molecules as either protein or peptide, this would include pro-inflammatory cytokines such as Interferon, particular Interferon alpha, GM-CSF, IL-1 alpha, IL-1 beta, TGF-alpha and TGF-beta, Th1 inducers such as interferon gamma, IL-2, IL-12, IL-15, IL-18 and IL-21, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L, other immunostimulatory targeting ligands such as CTLA-4 and L-selectin, apoptosis stimulating proteins and peptides such as Fas, (49), synthetic lipid based adjuvants, such as vaxfectin, (Reyes et al., ‘Vaxfectin enhances antigen specific antibody titres and maintains Th1 type immune responses to plasmid DNA immunization’, Vaccine 19: 3778-3786) squalene, alpha-tocopherol, polysorbate 80, DOPC and cholesterol, endotoxin, [LPS], Beutler, B., ‘Endotoxin, ‘Toll-like receptor 4, and the afferent limb of innate immunity’, Current Opinion in Microbiology 3: 23-30 (2000)); CpG oligo- and di-nucleotides, Sato, Y. et al., ‘Immunostimulatory DNA sequences necessary for effective intradermal gene immunization’, Science 273 (5273): 352-354 (1996). Hemmi, H. et al., ‘A Toll-like receptor recognizes bacterial DNA’, Nature 408: 740-745, (2000) and other potential ligands that trigger Toll receptors to produce Th1-inducing cytokines, such as synthetic Mycobacterial lipoproteins, Mycobacterial protein p19, peptidoglycan, teichoic acid and lipid A. Other bacterial immunostimulatory proteins such as Cholera Toxin, E. coli Toxin and mutant toxoids thereof can be utilised.

Certain preferred adjuvants for eliciting a predominantly Th1-type response to a protein antigen include for example, a Lipid A derivative such as monophosphoryl lipid A, or preferably 3-de-O-acylated monophosphoryl lipid A. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins.

Also provided are the use of a polynucleotide according to the invention, or of a vector according to the invention, in the treatment or prophylaxis of MUC-1 expressing tumour or metastases.

The present invention also provides methods of treating or preventing MUC-1 expressing tumour, any symptoms or diseases associated therewith including metastases, comprising administering an effective amount of a polynucleotide, a vector or a pharmaceutical composition according to the invention. Administration of a pharmaceutical composition may take the form of one or more individual doses, for example in a “prime-boost” therapeutic vaccination regime. In certain cases the “prime” vaccination may be via particle mediated DNA delivery of a polynucleotide according to the present invention, preferably incorporated into a plasmid-derived vector and the “boost” by administration of a recombinant viral vector comprising the same polynucleotide sequence, or boosting with the protein of the invention in adjuvant. Conversely the priming may be with the viral vector or with a protein formulation typically a protein formulated in adjuvant and the boost a DNA vaccine of the present invention.

As discussed above, the present invention includes expression vectors that comprise the nucleotide sequences of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. Molecular Cloning: a Laboratory Manual. 2^{nd} Edition. CSH Laboratory Press. (1989).

Preferably, a polynucleotide of the invention, or for use in the invention in a vector, is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be, for example, plasmids, artificial chromosomes (e.g. BAC, PAC, YAC), virus or phage vectors provided with an origin of replication, optionally a promoter for the expression of the polynucleotide and optionally a regulator of the promoter. The vectors may contain one or more selectable marker genes, for example an ampicillin or kanamycin resistance gene in the case of a bacterial plasmid or a resistance gene for a fungal vector. Vectors may be used in vitro, for example for the production of DNA or RNA or used to transfect or transform a host cell, for example, a mammalian host cell e.g. for the production of protein encoded by the vector. The vectors may also be adapted to be used in vivo, for example in a method of DNA vaccination or of gene therapy.

Promoters and other expression regulation signals may be selected to be compatible with the host cell for which expression is designed. For example, mammalian promoters include the metallothionein promoter, which can be induced in response to heavy metals such as cadmium, and the β-actin promoter. Viral promoters such as the SV40 large T antigen promoter, human cytomegalovirus (CMV) immediate early (IE) promoter, rous sarcoma virus LTR promoter, adenovirus promoter, or a HPV promoter, particularly the HPV upstream regulatory region (URR) may also be used. All these promoters are well described and readily available in the art.

A preferred promoter element is the CMV immediate early promoter devoid of intron A, but including exon 1. Accordingly there is provided a vector comprising a polynucleotide of the invention under the control of HCMV IE early promoter.

Examples of suitable viral vectors include herpes simplex viral vectors, vaccinia or alpha-virus vectors and retroviruses, including lentiviruses, adenoviruses and adeno-associated viruses. Gene transfer techniques using these viruses are known to those skilled in the art. Retrovirus vectors for example may be used to stably integrate the polynucleotide of the invention into the host genome, although such recombination is not preferred. Replication-defective adenovirus vectors by contrast remain episomal and therefore allow transient expression. Vectors capable of driving expression in insect cells (for example baculovirus vectors), in human cells or in bacteria may be employed in order to produce quantities of the HIV protein encoded by the polynucleotides of the present invention, for example for use as subunit vaccines or in immunoassays. The polynucleotides of the invention have particular utility in viral vaccines as previous attempts to generate full-length vaccinia constructs have been unsuccessful.

Bacterial vectors, such as attenuated Salmonella or Listeria may also be used. The polynucleotides according to the invention have utility in the production by expression of the encoded proteins, which expression may take place in vitro, in vivo or ex vivo. The nucleotides may therefore be involved in recombinant protein synthesis, for example to increase yields, or indeed may find use as therapeutic agents in their own right, utilised in DNA vaccination techniques. Where the polynucleotides of the present invention are used in the production of the encoded proteins in vitro or ex vivo, cells, for example in cell culture, will be modified to include the polynucleotide to be expressed. Such cells include transient, or preferably stable mammalian cell lines. Particular examples of cells which may be modified by insertion of vectors encoding for a polypeptide according to the invention include mammalian HEK293T, CHO, HeLa, 293 and COS cells. Preferably the cell line selected will be one which is not only stable, but also allows for mature glycosylation and cell surface expression of a polypeptide. Expression may be achieved in transformed oocytes. A polypeptide may be expressed from a polynucleotide of the present invention, in cells of a transgenic non-human animal, preferably a mouse. A transgenic non-human animal expressing a polypeptide from a polynucleotide of the invention is included within the scope of the invention.

The invention further provides a method of vaccinating a mammalian subject which comprises administering thereto an effective amount of such a vaccine or vaccine composition. Most preferably, expression vectors for use in DNA vaccines, vaccine compositions and immunotherapeutics will be plasmid vectors.

DNA vaccines may be administered in the form of “naked DNA”, for example in a liquid formulation administered using a syringe or high pressure jet, or DNA formulated with liposomes or an irritant transfection enhancer, or by particle mediated DNA delivery (PMDD). All of these delivery systems are well known in the art. The vector may be introduced to a mammal for example by means of a viral vector delivery system.

The compositions of the present invention can be delivered by a number of routes such as intramuscularly, subcutaneously, intraperitonally, intravenously. Or via the mucosal route, e.g intranasally.

In a preferred embodiment, the composition is delivered intradermally. In particular, the composition is delivered by means of a gene gun (particularly particle bombardment) administration techniques which involve coating the vector on to a bead (eg gold) which are then administered under high pressure into the epidermis; such as, for example, as described in Haynes et al, J Biotechnology 44: 37-42 (1996).

In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles are preferably gold beads of a 0.4-4.0 μm, more preferably 0.6-2.0 μm diameter and the DNA conjugate coated onto these and then encased in a cartridge or cassette for placing into the “gene gun”.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

The nucleic acid vaccine may also be delivered by means of micro needles, which may be coated with a composition of the invention or delivered via the micro-needle from a reservoir.

The vectors which comprise the nucleotide sequences encoding antigenic peptides are administered in such amount as will be prophylactically or therapeutically effective. The quantity to be administered, is generally in the range of one picogram to 1 milligram, preferably 1 picogram to 10 micrograms for particle-mediated delivery, and 10 micrograms to 1 milligram for other routes of nucleotide per dose. The exact quantity may vary considerably depending on the weight of the patient being immunised and the route of administration.

It is possible for the immunogen component comprising the nucleotide sequence encoding the antigenic peptide, to be administered on a once off basis or to be administered repeatedly, for example, between 1 and 7 times, preferably between 1 and 4 times, at intervals between about 1 day and about 18 months. Once again, however, this treatment regime will be significantly varied depending upon the size of the patient, the disease which is being treated/protected against, the amount of nucleotide sequence administered, the route of administration, and other factors which would be apparent to a skilled medical practitioner. The patient may receive one or more other anti cancer drugs as part of their overall treatment regime.

Suitable techniques for introducing the naked polynucleotide or vector into a patient also include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical simulation, electroporation and microseeding which is described in U.S. Pat. No. 5,697,901.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered.

A nucleic acid sequence of the present invention may also be administered by means of transformed cells. Such cells include cells harvested from a subject. The naked polynucleotide or vector of the present invention can be introduced into such cells in vitro and the transformed cells can later be returned to the subject. The polynucleotide of the invention may integrate into nucleic acid already present in a cell by homologous recombination events. A transformed cell may, if desired, be grown up in vitro and one or more of the resultant cells may be used in the present invention. Cells can be provided at an appropriate site in a patient by known surgical or microsurgical techniques (e.g. grafting, micro-injection, etc.)

The invention will now be illustrated by reference to the following examples:

### EXAMPLES

Introduction

The experiments demonstrate the use of the Mycobacterium tuberculosis heat-shock protein 70 (HSP70) to enhance the cellular immune response to MUC-1 derivative. A series of constructs have been generated in which the HSP70 gene is fused to either the N- or C-terminus of MUC1. Significant differences both in the stability and immunogenicity of the various fusion constructs have been observed. Fusion to HSP70 improves the kinetics and functionality of immune response to MUC1.

Materials & Methods

1. Construction of M. tuberculosis HSP70 Expression Vector for Fusion of N-Terminal Expression Cassettes

The starting vectors JNW340, JNW358, JNW640 and JNW656 are described in the UK patent application number 02/12046.47. A schematic of the relationship between all the constructs is shown in Appendix C.

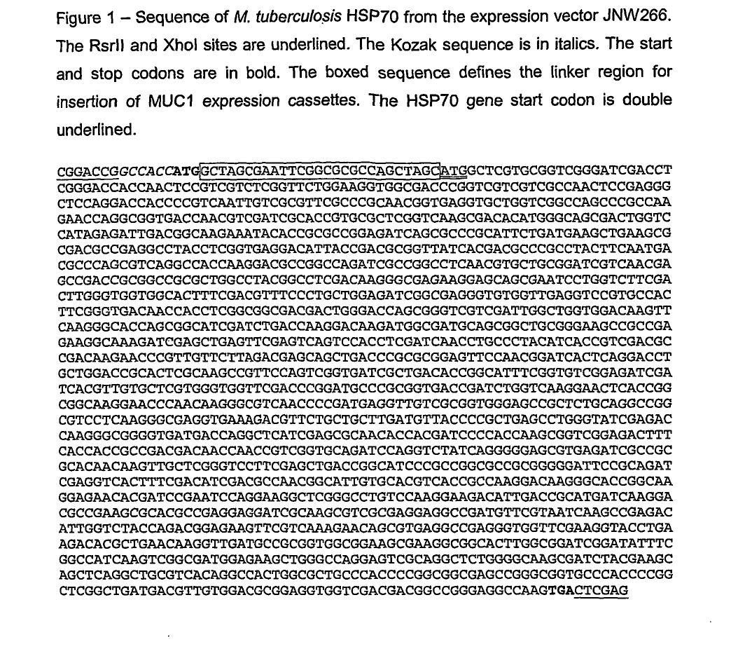

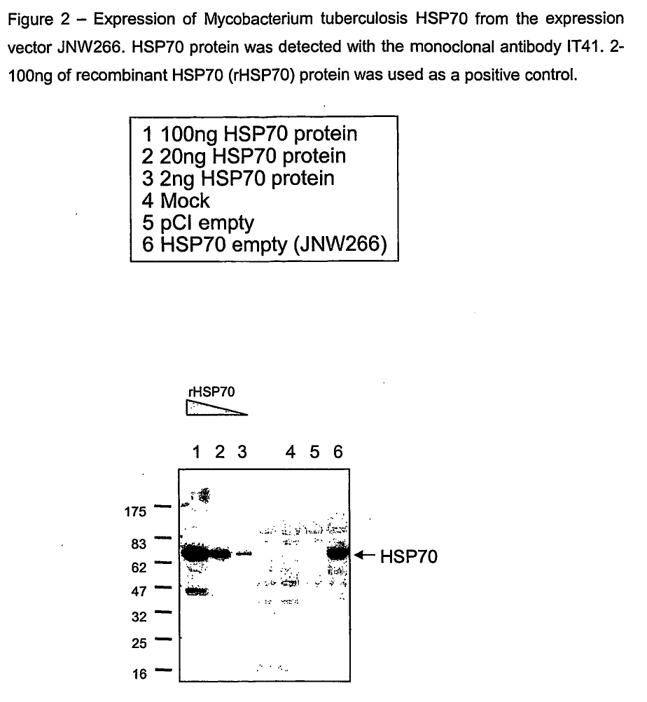

The M. tuberculosis (MTB) HSP70 gene was PCR amplified from the genomic DNA of strain CSU93 (GSK, Stevenage, UK) using PCR primers 2039HSP70 and 2041HSP70 (see Appendix A). The PCR fragment was restricted with XbaI and XhoI, ligated into the vector pVAC (restricted NheI-XhoI) and sequence verified using primers 2042HSP70-2059HSP70. The validated construct was labelled JNW266. This construct contains the full-length HSP70 gene with NheI, EcoRI and AscI cloning sites for insertion of fusion cassettes at its N-terminus (see FIG. 1 for full sequence). Expression of HSP70 was confirmed in vitro using a transient transfection assay. A Western blot of a total cell lysate with IT41 (World Health Organisation), an anti-HSP70 monoclonal antibody, revealed the presence of a signal band of ˜70 kDa, coincident in size with MTB HSP70 protein (see FIG. 2).

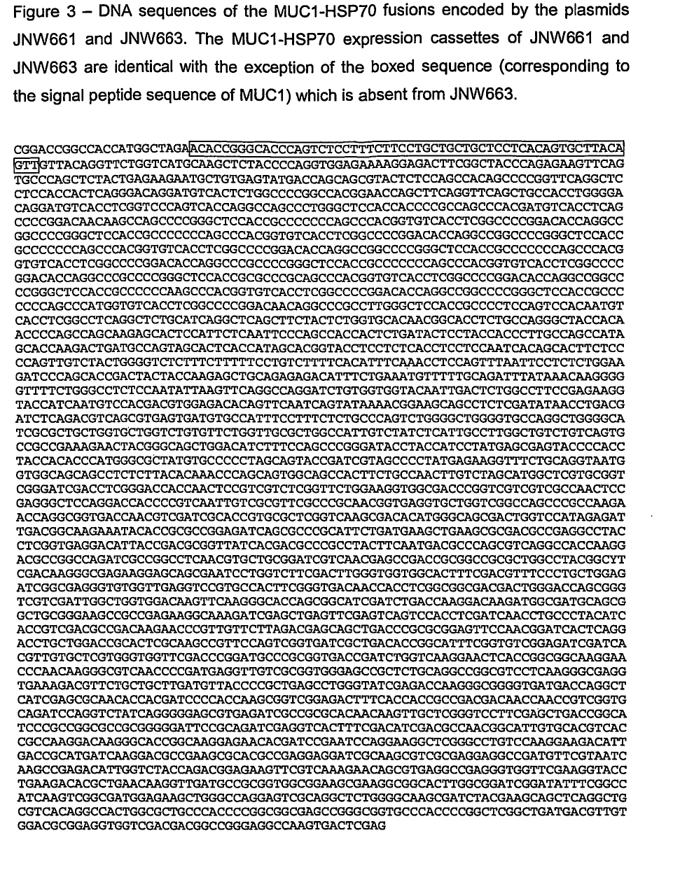

The 7× VNTR MUC1 expression cassettes with and without signal peptide sequence were isolated from plasmids JNW640 (+signal peptide) and JNW645 (−signal peptide) by XbaI digest and ligated between the NheI sites of JNW266, generating plasmids JNW661 (+signal peptide) and JNW663 (−signal peptide) respectively. The FL-MUC1 cassette was isolated in a similar manner from plasmid JNW340 (+signal peptide) and inserted between the NheI sites of JNW266, generating plasmid JNW381. The sequences of the MUC1-HSP70 constructs (JNW661 and JNW663) are shown in FIG. 3. Schematics of all constructs are shown in Appendix B.

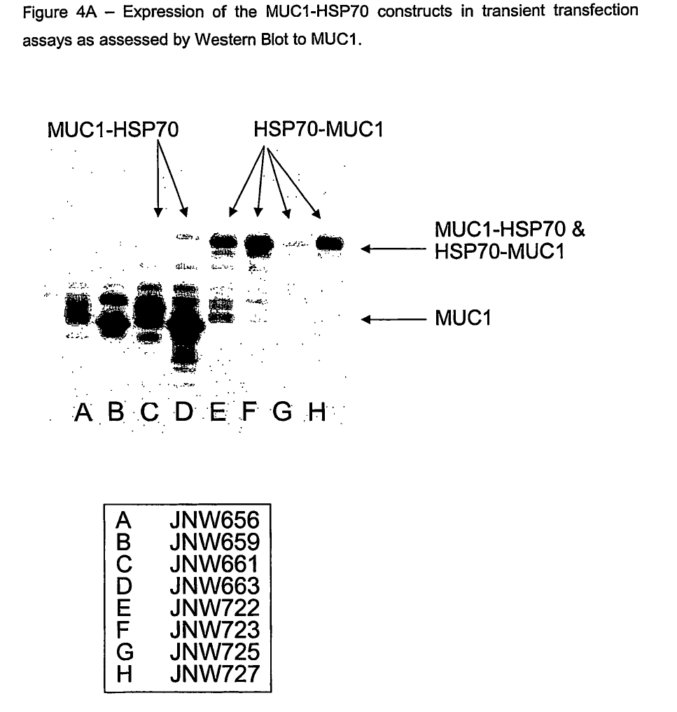

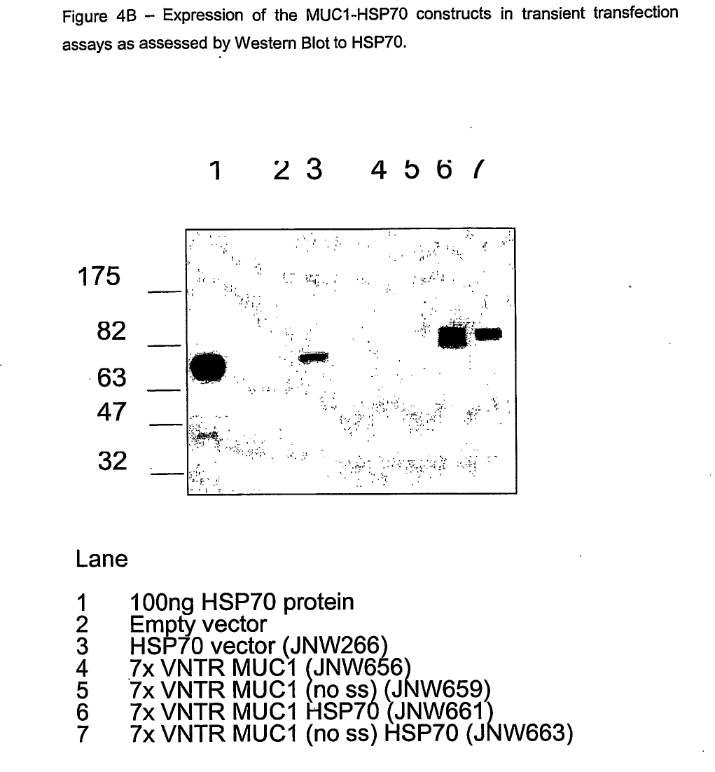

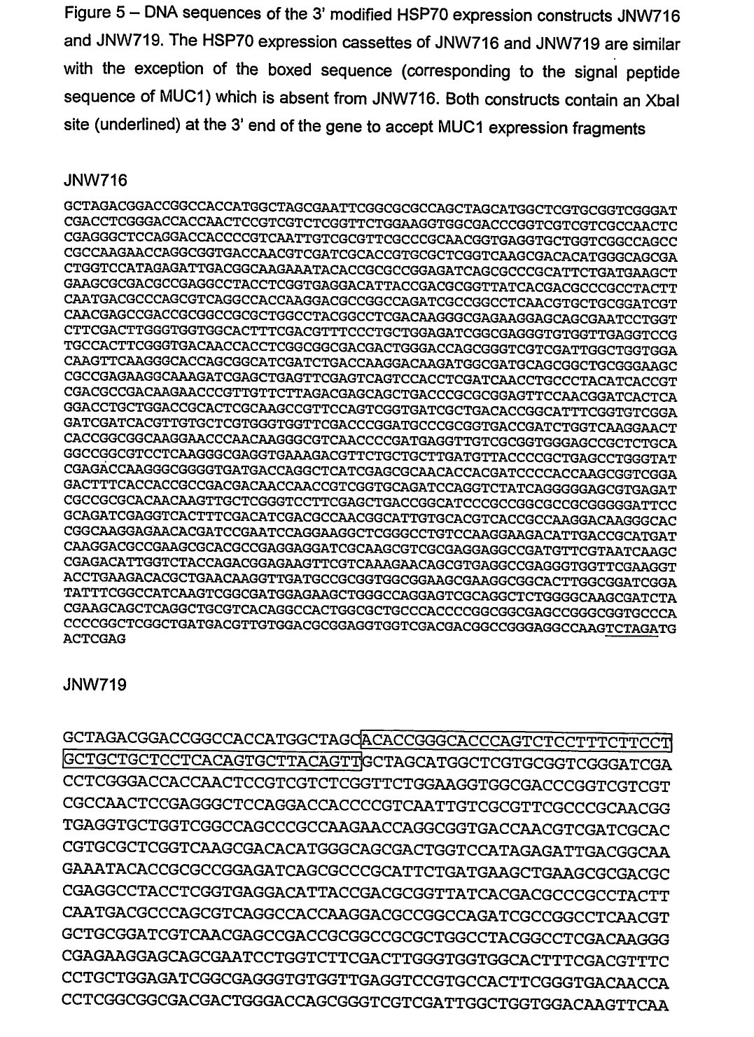

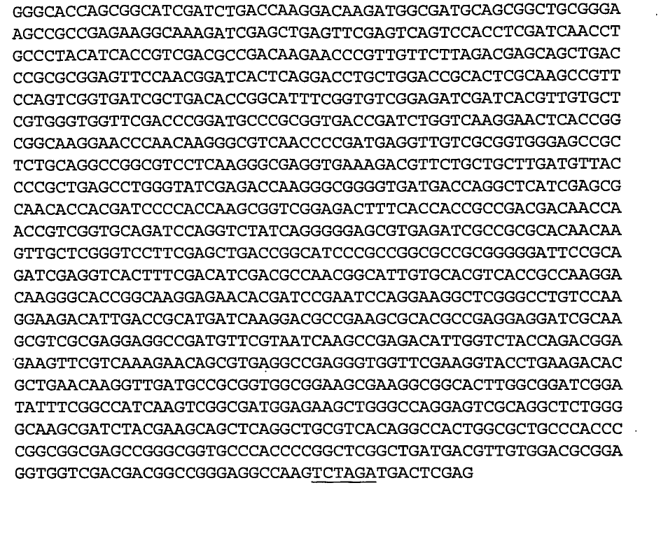

Transient transfection of JNW661 and JNW663 into CHO cells shows that the MUC1-HSP70 fusion protein is unstable in vitro, with the fusion protein cleaving into two fragments (FIG. 4A and 4B). The size of the MUC1 and HSP70 fragments suggest that the cleavage site occurs within the C-terminal section of MUC1 and is consistent with recent reports of a cleavage site in MUC1 which is subjected to co-translational proteolytic processing (Parry et al. (2001) Biochem. Biophys. Res. Com. 283: 715-720).

2. Construction of M. tuberculosis HSP70 Expression Vectors for Fusion of C-Terminal Expression Cassettes

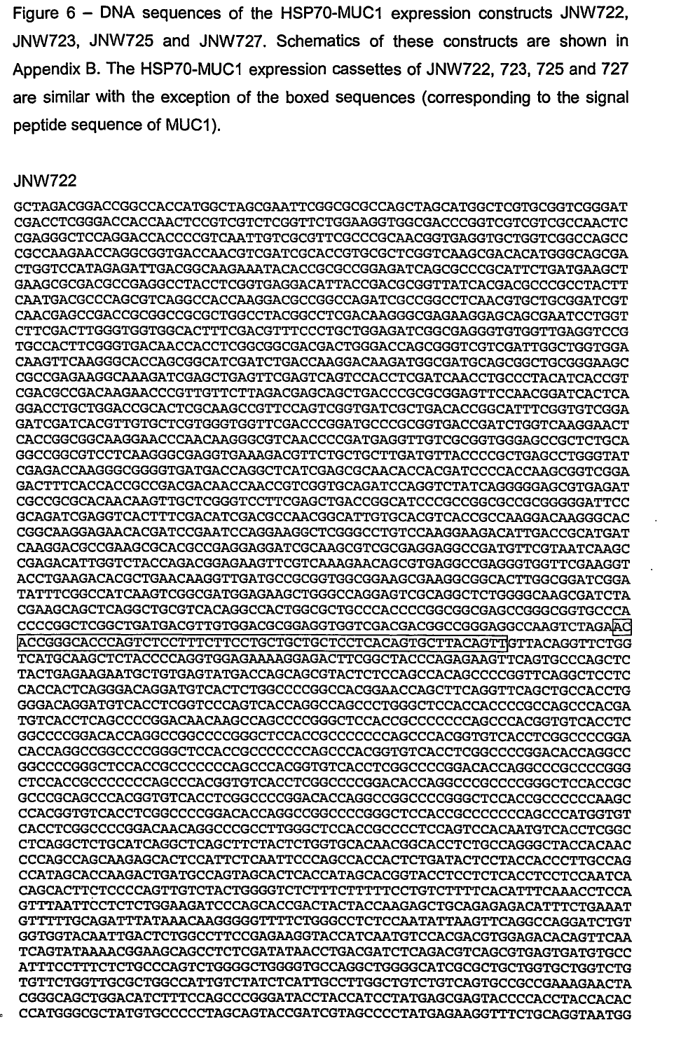

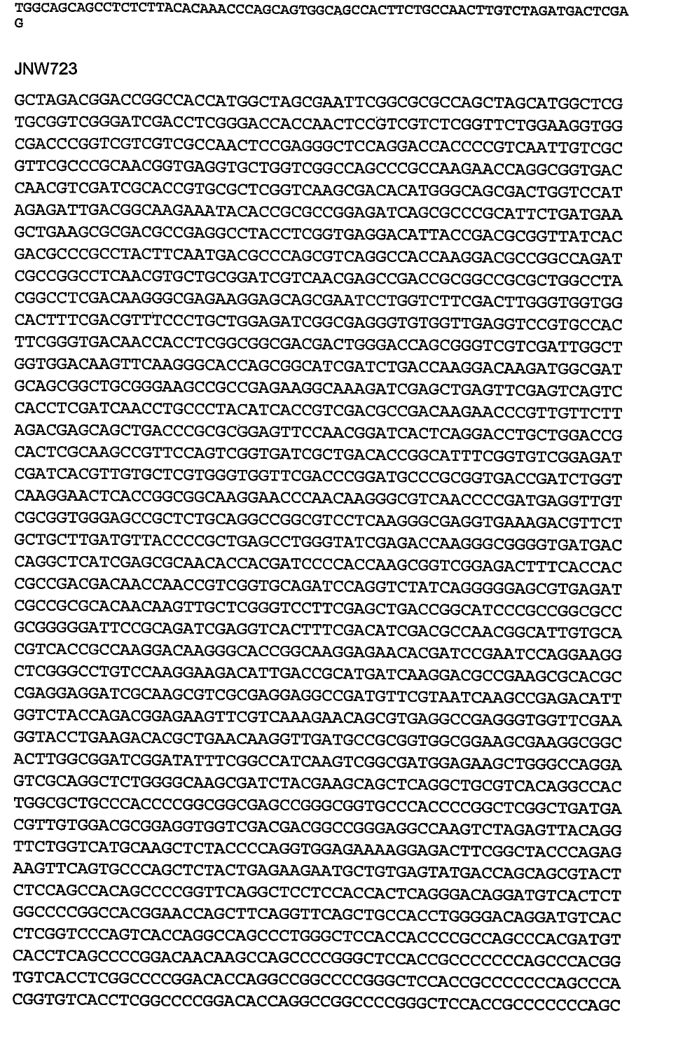

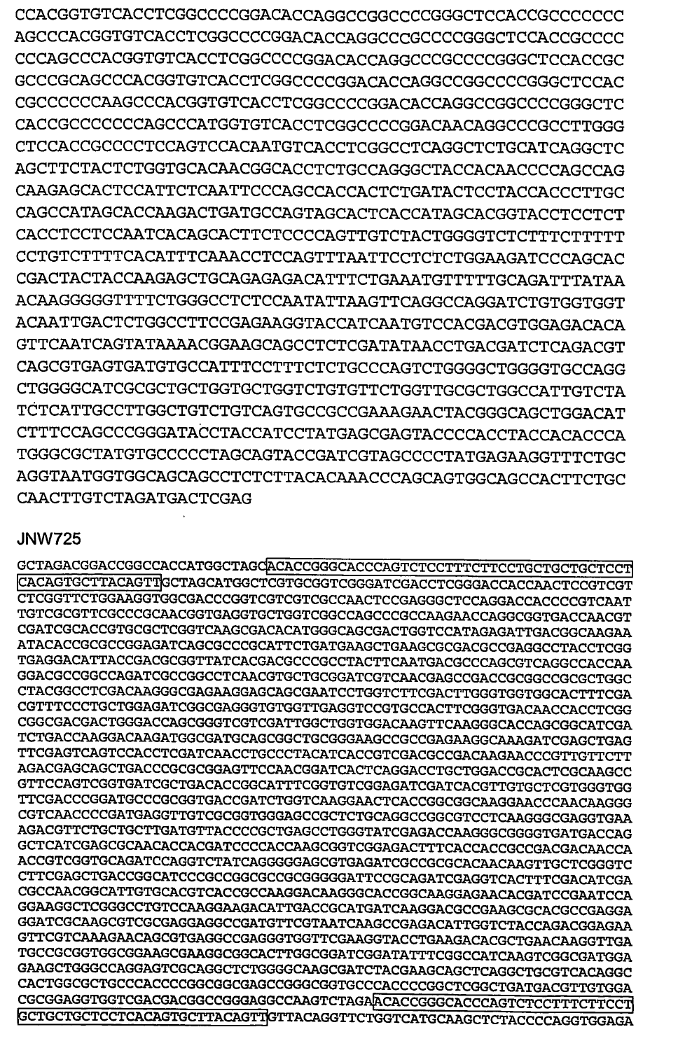

In an attempt to improve the stability of the MUC1-HSP70 fusion protein, the order of the two components was switched. However, in these constructs the signal peptide sequence of MUC1, important for directing MUC1 to the correct intracellular processing pathway, will be hidden in the central section of the fusion protein. In an attempt to alleviate this problem, two different vectors were constructed for fusion of C-terminal MUC1 expression cassettes. The first contains the HSP70 with a MUC1 signal peptide sequence at the N-terminus, the second vector is without the signal peptide sequence. To insert the MUC1 signal peptide sequence at the N-terminus of HSP70, a oligonucleotide linker was constructed from primers 2077MUC1 and 2078MUC1 and ligated between the NheI sites of JNW266, generating plasmid JNW708. The C-terminus of HSP70 of plasmids JNW266 and JNW708 was re-engineered to accept MUC1 expression cassettes by PCR amplifying the C-terminus of HSP70 with primers 2075MUC1 and 2076MUC1. The PCR fragment was restricted with BIpI and XhoI and ligated into JNW266 and JNW708 previously restricted with BIpI and XhoI, generating the plasmids JNW716 and JNW719 respectively (FIG. 6). The 7× VNTR MUC1 expression cassettes±signal peptide sequence were isolated on XbaI fragments from JNW656 (+signal peptide) and JNW659 (−signal peptide) and cloned into the XbaI sites of JNW716 and JNW719, generating four new vectors—JNW722, JNW723, JNW725 and JNW727. All four vectors have MUC1 at the C-terminus of HSP70 but have the signal peptide at different positions (Shown in Appendix B).

Transient transfection analysis of the plasmids JNW722, JNW723 and JNW727 confirms that the fusion protein is stable in vitro (see FIG. 4A). No expression of JNW725 was detected by Western blot. In terms of MUC1 expression at the surface of CHO cells (as determined by FACS analysis following staining with the anit-MUC1 antibody ATR1), plasmids JNW722 and JNW727 showed the best levels of expression and were selected for in vivo analysis.

Testing of Constructs: Materials

3.1 B16F0 and B16F0-MUC1 Tumour Cells

B16F0 (murine metastatic melanoma) transfected with an expression vector for the human cDNA MUC1 were obtained from GlaxoWellcome U.S. Cells were cultivated as adherent monolayers in DMEM supplemented with 10% heat inactivated fetal calf serum, 2 mM L-glutamine, 100 U/ml penicillin, 100 μg/ml streptomycin and 1 mg/ml of neomycin antibiotic (G148). For use in ELISPOT assays cells were removed from flasks using Versene and irradiated (16,000 Rads).

3.2 Cutaneous Gene Gun Immunisation

Plasmid DNA was precipitated onto 2 μm diameter gold beads using calcium chloride and spermidine. Loaded beads were coated onto Tefzel tubing as described (Eisenbraum et al, 1993; Pertmer et al, 1996). Particle bombardment was performed using the Accell gene delivery system (PCT WO 95/19799). For each plasmid, female C56BI/6 mice were immunised with 3 administrations of plasmid on days 0, 21 and 42. Each administration consisted of two bombardments with DNA/gold, providing a total dose of approximately 4-5 μg of plasmid.

3.3 Tumour Cell Injection

0.5×10⁶ or 1.0×10⁶ tumour cells were subcutaneously injected in the right flank of anaesthetized animals two weeks after the last immunisation. Tumour growth was monitored twice a week using vernier calipers in two dimensions. Tumour volumes were calculated as (a×b²)/2, where a represents the largest diameter and b the smallest diameter. The experimental endpoint (death) was defined as the time point at which tumour diameter reached 15 mm.

3.4 ELISPOT Assays for T Cell Responses to the MUC1 Gene Product Preparation of Splenocytes

Spleens were obtained from immunised animals at 7-14 days post boost. Spleens were processed by grinding between glass slides to produce a cell suspension. Red blood cells were lysed by ammonium chloride treatment and debris was removed to leave a fine suspension of splenocytes. Cells were resuspended at a concentration of 8×10⁶/ml in RPMI complete media for use in ELISPOT assays.

ELISPOT Assay

Plates were coated with 15 μg/ml (in PBS) rat anti mouse IFNγ or rat anti mouse IL-2 (Pharmingen). Plates were coated overnight at +4° C. Before use the plates were washed three times with PBS. Splenocytes were added to the plates at 4×10⁵ cells/well. Peptides SAPDNRPAL (SAP), TSAPDNRPA (TSA) and PTTLASHS (PTT) were used in assays at a final concentration of 10 nM, 1□M and 1□M respectively. Peptides were obtained from Genemed Synthesis. Irradiated tumour cells B16 and B16-MUC1 were used at a tumour cell: effector ratio of 1:4. ELISPOT assays were carried out in the presence of either IL-2 (10 ng/ml), IL-7 (10 ng/ml) or no cytokine. Total volume in each well was 200 μl. Plates containing peptide stimulated cells were incubated for 16 hours in a humidified 37° C. incubator while those containing tumour cells as stimulators were incubated for 40 hours.

Development of ELISPOT Assay Plates.

Cells were removed from the plates by washing once with water (with 1 minute soak to ensure lysis of cells) and three times with PBS. Biotin conjugated rat anti mouse IFNγ or IL-2 (Phamingen) was added at 1 μg/ml in PBS. Plates were incubated with shaking for 2 hours at room temperature. Plates were then washed three times with PBS before addition of Streptavidin alkaline phosphatase (Caltag) at 1/1000 dilution. Following three washes in PBS spots were revealed by incubation with BCICP substrate (Biorad) for 15-45 mins. Substrate was washed off using water and plates were allowed to dry. Spots were enumerated using an image analysis system devised by Brian Hayes, Asthma Cell Biology unit, GSK.

3.5 CTL Assays

Bulk Cultures to Generate Effectors

Stimulator cells were irradiated at 3000 rad and resuspended at 5×10⁶/ml (stimulators may be peptide pulsed splenocytes or transfectants as appropriate). Stimulator cells were incubated at a ratio of 1:4 with effector cells (splenocytes), either in tissue culture flasks or plates in the presence of IL-2 (10 ng/ml) for at least 5-7 days before use in CTL assay. Peptides were added at the following concentrations (SAP at 40 nM, PTT at 4 μM and TSA at 4 μM)

Effector Cells Preparation

The effector cells were harvested from bulk cultures described above after 5-7 days, washed three times in medium and resuspended at 2.5×10⁶/ml in RPMI complete medium. 100 μl of effector cells was aliquoted into U-bottomed plates at decreasing cell densities.

Europium Labelling of Target Cells

The target cells were washed in complete medium then Hepes buffer and resuspended to 1×10⁷/ml in ice cold labelling buffer. The cells were labelled for 40 minutes on ice with frequent shaking. 9 ml of ice-cold repair buffer was added to the cells and incubated on ice for a further 5 minutes. The cells were then washed three times in ice-cold repair buffer followed by two times in cold culture medium. The cells were finally resuspended at 1×10⁷/ml in warm culture medium. The target cells were then pulsed with peptide (SAP at 160 nM, PTT and TSA at 10 μM) for 1 hour at 37° C. as required. Prior to use, the pulsed target cells were washed twice in warm culture medium and resuspended at a concentration of 5×10⁴/ml in warm culture medium

Assay

100 μl target cells was added to all wells of 96 well plate already containing effector cells. The plate was spun at 1000 rpm for 2 mins and then incubated at 37° C. At each timepoint, 20 μl was collected and transfered into a separate 96-well ELISA plate. 200 μl of Enhancement solution was added to each well. The plate was placed on shaker for 5 mins and read on Wallac Victor using the Europium programme.
% specific cytotoxicity=(test release−spontaneous release)/(max release−spontaneous release)×100
Reagents
RPMI Complete:

RPMI+10% FCS+2 mM glutamine+50□M 2-mercaptoethanol

Complete Hepes buffer (pH 7.4)

50 mM HEPES, 83 mM NaCl, 5 mM KCl, 2 mM MgCl₂

Europium Labelling Buffer

To 200 ml Hepes complete add: 600 mM EuCl₃, 3 mM DTPA, 5 mg Dextran sulphate

Repair Buffer (pH 7.4)

To 500 mls Hepes complete add: 2 mM CaCl₂, 10 mM D-glucose

3.6 Flow Cytometry to Detect IFNγ Production from T Cells in Response to Peptide Stimulation.

Splenocytes were resuspended at 4×10⁶/ml. Peptide was added at a final concentration of 10 μM and IL-2 at a final concentration of 10 ng/ml. Cells were incubated at 37° C. for 3 hours, Brefeldin A was added at 10 μg/ml, and incubation continued overnight. Cells were washed with FACS buffer (PBS+2.5% FCS+0.1% azide) and stained with anti CD4 Cychrome and anti CD8 FITC (Pharmingen). Cells were washed and fixed with Medium A from Caltag Fix and Perm kit for 15 mins followed by washing and addition of anti IFNγ PE (Pharmingen) diluted in Medium B from the Fix and Perm kit. After 30 mins incubation cells were washed and analysed using a FACSCAN. A total of 500,000 cells were collected per sample and subsequently CD4 and CD8 cells were gated to determine the populations of cells secreting IFNγ in response to each peptide.

3.7 Transient Transfection Assays

MUC1 expression from various DNA constructs was analysed by transient transfection of the plasmids into CHO (Chinese hamster ovary) cells followed by either Western blotting on total cell protein, or by flow cytometric analysis of cell membrane expressed MUC1. Transient transfections were performed with the Transfectam reagent (Promega) according to the manufacturer's guidelines. In brief, 24-well tissue culture plates were seeded with 5×10⁴ CHO cells per well in 1 ml DMEM complete medium (DMEM, 10% FCS, 2 mM L-glutamine, penicillin 100 IU/ml, streptomycin 100 μg/ml) and incubated for 16 hours at 37° C. 0.5 μg DNA was added to 25 μl of 0.3M NaCl (sufficient for one well) and 2 μl of Transfectam was added to 25 μl of Milli-Q. The DNA and Transfectam solutions were mixed gently and incubated at room temperature for 15 minutes. During this incubation step, the cells were washed once in PBS and covered with 150 μl of serum free medium (DMEM, 2 mM L-glutamine). The DNA-Transfectam solution was added drop wise to the cells, the plate gentle shaken and incubated at 37° C. for 4-6 hours. 500 μl of DMEM complete medium was added and the cells incubated for a further 48-72 hours at 37° C.

3.8 Flow Cytometric Analysis of CHO Cells Transiently Transfected with MUC1 Plasmids

Following transient transfection, the CHO cells were washed once with PBS and treated with a Versene (1:5000)/0.025% trypsin solution to transfer the cells into suspension. Following trypsinisation, the CHO cells were pelleted and resuspended in FACS buffer (PBS, 4% FCS, 0.01% sodium azide). The primary antibody, ATR1 was added to a final concentration of 15 μg/ml and the samples incubated on ice for 15 minutes. Control cells were incubated with FACS buffer in the absence of ATR1. The cells were washed three times in FACS buffer, resuspended in 100 μl FACS buffer containing 10 μl of the secondary antibody goat anti-mouse immunoglobulins FITC conjugated F(ab′)₂ (Dako, F0479) and incubated on ice for 15 minutes. Following secondary antibody staining, the cells were washed three times in FACS buffer. FACS analysis was performed using a FACScan (Becton Dickinson). 1000-10000 cells per sample were simultaneously measured for FSC (forward angle light scatter) and SSC (integrated light scatter) as well as green (FL1) fluorescence (expressed as logarithm of the integrated fluorescence light). Recordings were made excluding aggregates whose FCS were out of range. Data were expressed as histograms plotted as number of cells (Y-axis) versus fluorescence intensity (X-axis).

3.9 Western Blot Analysis of CHO Cells Transiently Transfected with MUC1 Plasmids

The transiently transfected CHO cells were washed with PBS and treated with a Versene (1:5000)/0.025% trypsin solution to transfer the cells into suspension. Following trypsinisation, the CHO cells were pelleted and resuspended in 50□l of PBS. An equal volume of 2× TRIS-Glycine SDS sample buffer (Invitrogen) containing 50 mM DTT was added and the solution heated to 95° C. for 5 minutes. 1-20□l of sample was loaded onto a 4-20% TRIS-Glycine Gel 1.5 mm (Invitrogen) and electrophoresed at constant voltage (125V) for 90 minutes in 1× TRIS-Glycine buffer (Invitrogen). A pre-stained broad range marker (New England Biolabs, #P7708S) was used to size the samples. Following electrophoresis, the samples were transferred to Immobilon-P PVDF membrane (Millipore), pre-wetted in methanol, using an Xcell III Blot Module (Invitrogen), 1× Transfer buffer (Invitrogen) containing 20% methanol and a constant voltage of 25V for 90 minutes. The membrane was blocked overnight at 4° C. in TBS-Tween (Tris-buffered saline, pH 7.4 containing 0.05% of Tween 20) containing 3% dried skimmed milk (Marvel). The primary antibody (ATR1) was diluted 1:100 and incubated with the membrane for 1 hour at room temperature. Following extensive washing in TBS-Tween, the secondary antibody (#P0260, Dako) was diluted 1:2000 in TBS-Tween containing 3% dried skimmed milk and incubated with the membrane for one hour at room temperature. Following extensive washing, the membrane was incubated with Supersignal West Pico Chemiluminescent substrate (Pierce) for 5 minutes. Excess liquid was removed and the membrane sealed between two sheets of cling film, and exposed to Hyperfilm ECL film (AmershamPharmaciaBiotech) for 1-30 minutes. For probing for M. tuberculosis HSP70 expression, the primary antibody (IT41, WHO) was used at 1:100 to 1:500 followed by secondary antibody 1:1000 (#A9309, Sigma)

Results

4.1 Prophylactic Tumour Protection in Mice Immunised with Hsp70 Fusion Constructs

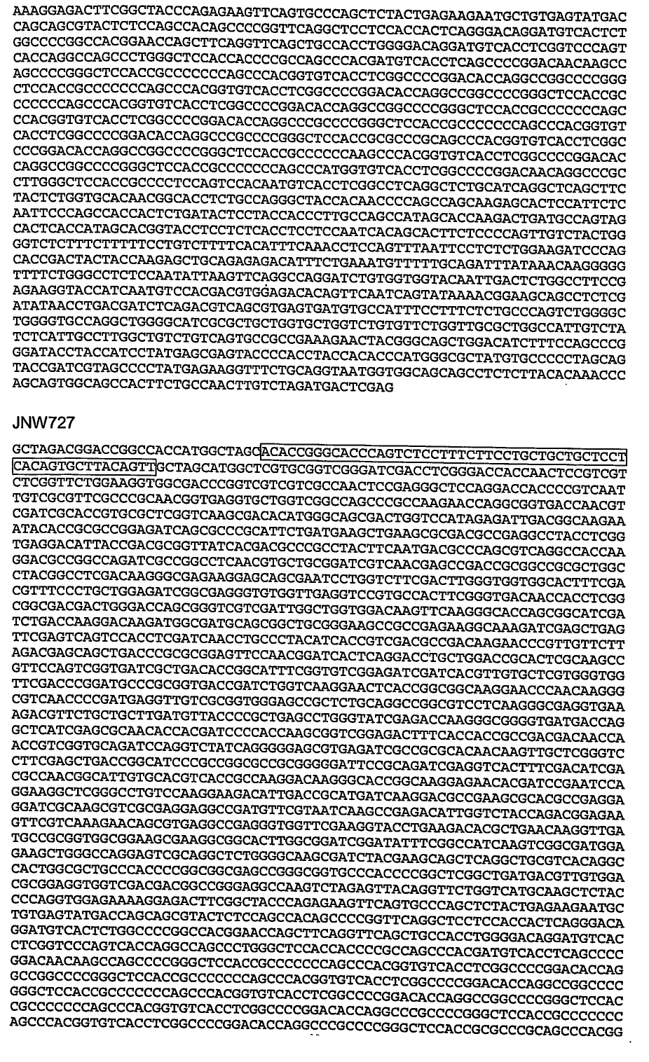

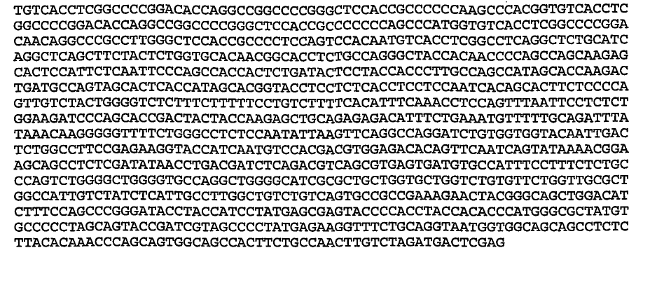

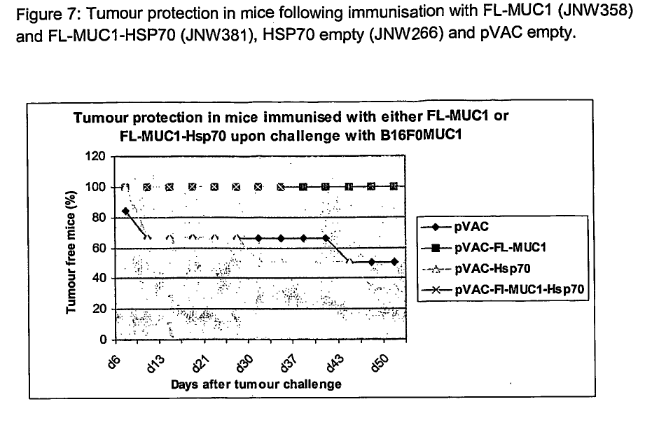

Mice were immunised with either FL-MUC1 (JNW358) or FL-MUC1-HSP70 (JNW381) and the relevant controls (pVAC empty vector and HSP70 empty vector, JNW266) at day 0, 21 and 42 and tumour cell injection was done at day 56. Tumours were measured over time as described in material and methods. As seen in FIG. 7, tumour protection was almost 100% with both FL-MUC1 and FL-MUC1-HSP70 constructs in contrast to 30 and 40% in the control groups.

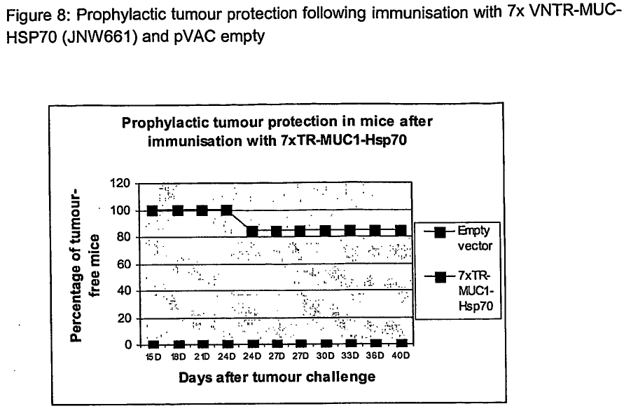

In another experiment, mice were immunised with either 7× VNTR-MUC1-HSP70 (JNW661) or pVAC empty vector at day 0 and tumour cells were implanted at day 21. Protection of mice in the vaccinated group was 85% whereas all mice in the control group had tumours (FIG. 8).

4.2 Cellular Responses in Mice Immunised with HSP70 Fusion Constructs

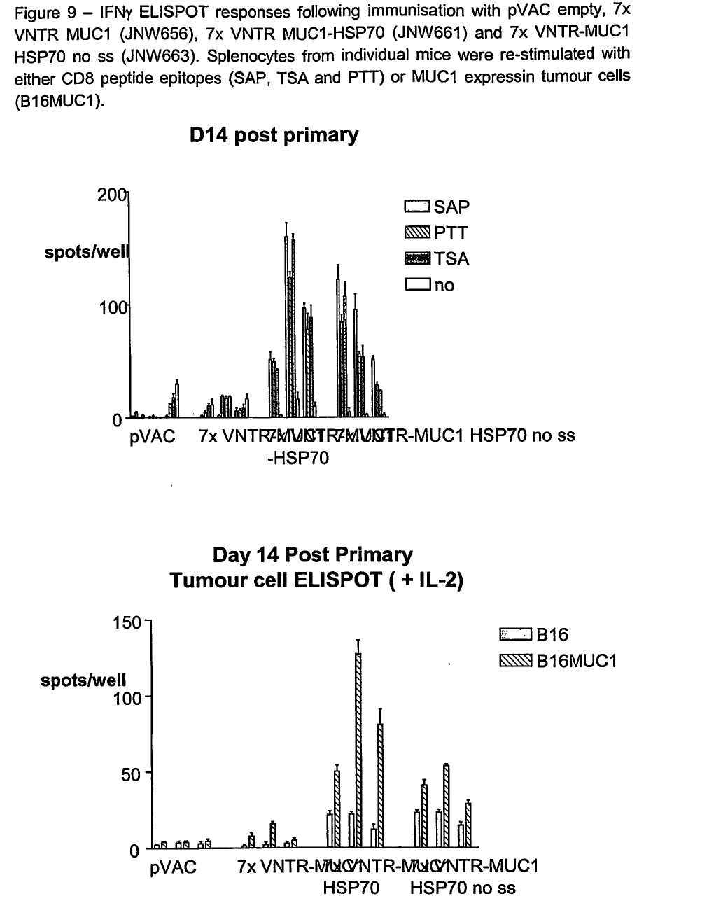

The cellular responses following immunisation with pVAC (empty vector), 7× VNTR MUC1 (JNW656), 7× VNTR MUC1-HSP70 (JNW661) and 7× VNTR MUC1-HSP70 no ss (JNW663) were assessed by ELISPOT following a primary immunisation by PMID at day 0. The assay was carried out at 14 days post primary using peptides (SAP, TSA and PTT peptides) and B16MUC1 tumour cells to re-stimulate the splenocytes. FIG. 9 shows that at day 14, whilst the 7× VNTR MUC1 construct induced no IFNγ secretion, both HSP70 fusion vectors (JNW661 and JNW663) induced good levels of IFNγ secretion in both the peptide and tumour cell ELISPOT assays.

4.3 Kinetics of Cellular Responses in Mice Immunised with HSP70 Fusion Constructs

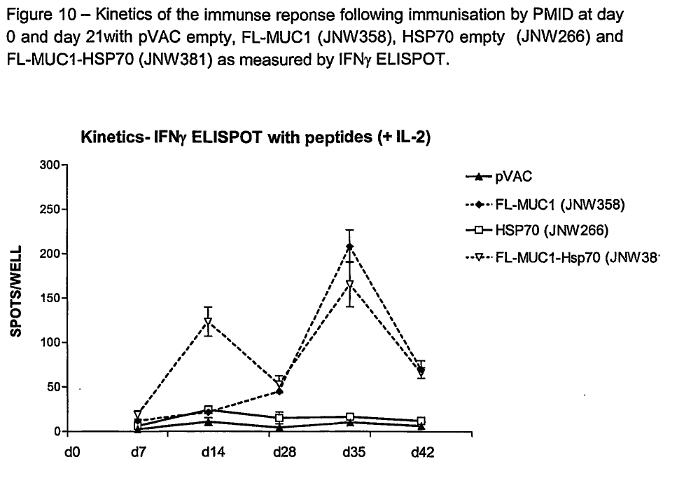

FIG. 10 shows the kinetics of the response of FL-MUC1 (JNW358) or FL-MUC1-HSP70 (JNW381) following immunisation by PMID at day 0 and day 21, as determined by IFNγ ELISPOT assays. Whilst the responses are very similar from day 21 onwards, the inclusion of the HSP70 component significantly enhances the primary response at day 14.

4.4 CTL Responses Following Immunisation with HSP70 Fusion Constructs

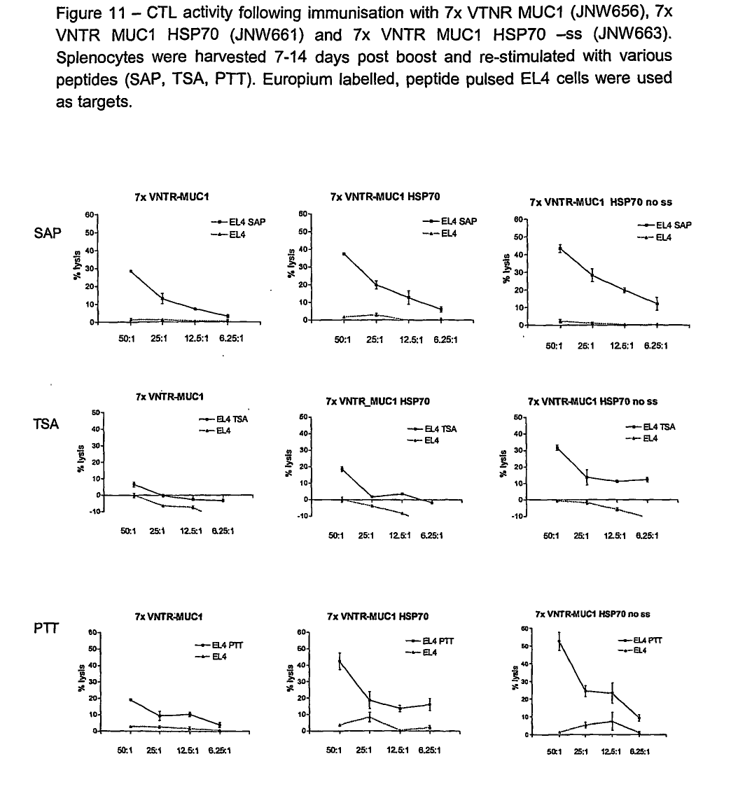

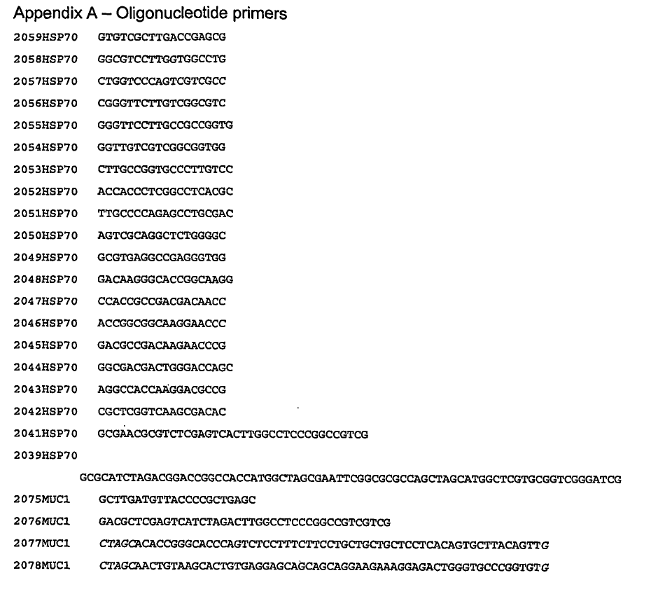

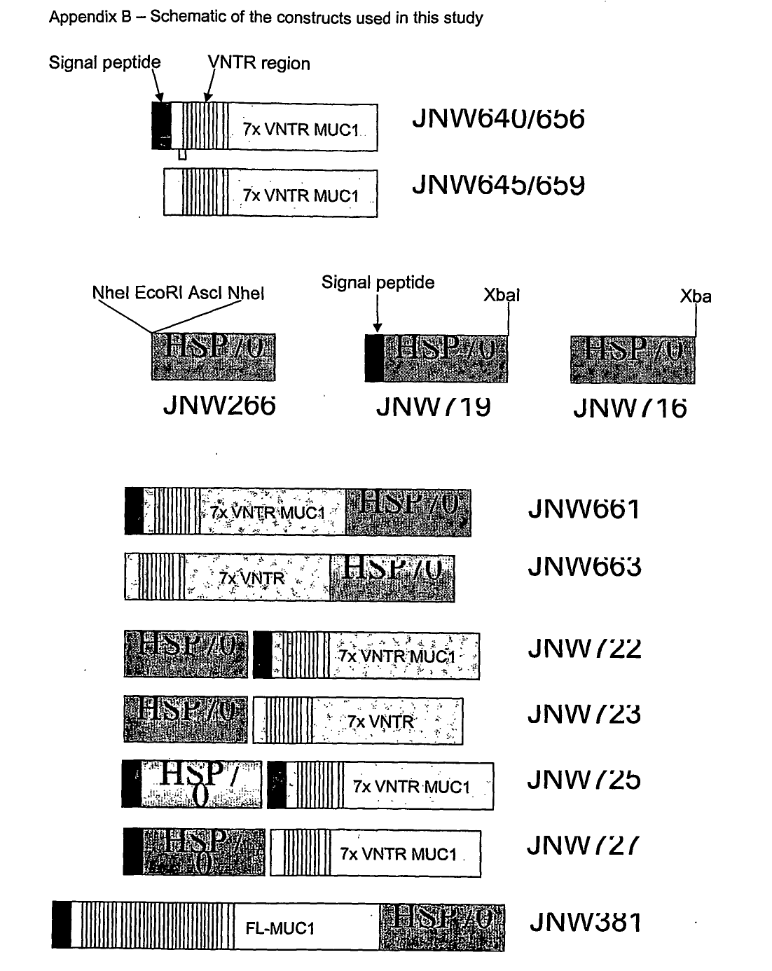

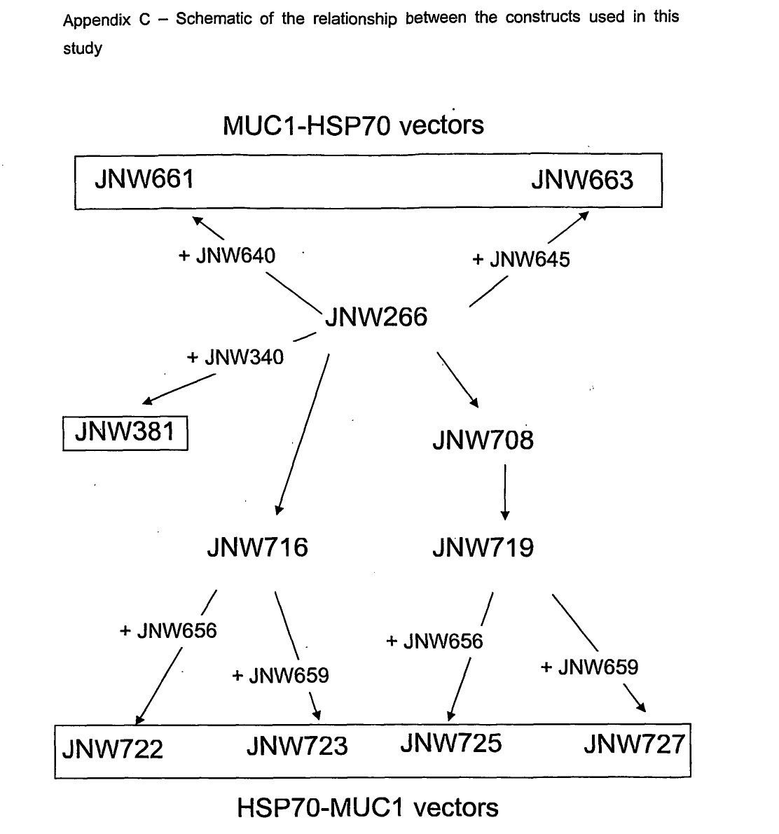

The cytolytic T lymphocyte (CTL) response was assessed following immunisation with the HSP70 fusion constructs. Lymphocytes were harvested 7-14 days post boost and re-stimulated with various MUC1 CD8 peptide epitopes (SAP, TSA, PTT). Following re-stimulation, the CTL activity of the effector cells was tested using peptide pulsed EL4 cells as targets in a europium release assay. FIG. 11 shows that whilst immunisation with 7× VNTR MUC1 induced CTL responses to all three peptides, the CTL activity was increased following immunisation with 7× VNTR-MUC1-HSP70±ss.

1. A nucleic acid molecule encoding a MUC-1 protein or derivative thereof which is capable of raising an immune response in vivo, said response being capable of recognising a MUC-1 expressing tumour, wherein the nucleic acid additional encodes a heat shock protein or fragment thereof.

2. A nucleic acid molecule as claimed in claim 1 wherein the heat shock protein is from a Mycobacterium.

3. A nucleic acid molecule as claimed in claim 1 wherein heat shock protein is HSP70.

4. A nucleic acid molecule encoding a MUC-1 derivative as claimed in claim 1 having less than 15 perfect repeat units.

5. A nucleic acid molecule as claimed in claim 4 having no perfect repeats.

6. A nucleic acid molecule as claimed in claim 1 of which is devoid of the signal sequence.

7. A nucleic acid molecule as claimed in claim 1 that encodes one or more of the sequence from the group:
FLSFHISNL; NSSLEDPSTDYYQELQRDISE; and NLTISDVSV.

8. A nucleic acid molecule as claimed in claim 1 additionally comprising a heterologous sequence that encodes a T-Helper epitope.

9. A nucleic acid molecule as claimed in claim 1 wherein the protein encoded by said molecule has the MUC-1 component at its C-terminus.

10. A nucleic acid molecule as claimed in claim 1 wherein the protein encoded by said molecule has the MUC-1 component at its n-terminus.

11. A nucleic acid molecule as claimed in claim 1 wherein the codon usage pattern is altered to more closely represent the codon bias of a highly expressed human gene.

12. A nucleic acid molecule as claimed in claim 1 that is a DNA molecule.

13. A protein encoded by a nucleic acid as claimed in claim 1.

14. A plasmid comprising the DNA molecule of claim 1.

15. A pharmaceutical composition comprising a nucleic acid as claimed in claim 1 and a pharmaceutical acceptable excipient, diluent or carrier.

16. A pharmaceutical composition as claimed in claim 15 wherein the carrier is microparticle.

17. A pharmaceutical composition as claimed in claim 16 wherein the microparticle is gold.

18. A pharmaceutical composition as claimed in claim 15 additionally comprising an adjuvant.

19. (canceled)

20. (canceled)

21. A method of treating or preventing tumours, comprising administering a safe and effective amount of a nucleic acid as claimed in claim 1.
